(19) United States
(12) Patent Application Publication
(10) Pub. No.: US 2021/0322465 A1
(43) Pub. Date: Oct. 21, 2021

(54) MAGNESIUM-CONTAINING FORMULATION AND USES THEREOF

(71) Applicant: TUDU HOLDINGS LTD., Tel-Aviv (IL)

(72) Inventor: David ZELINKER, Tel Aviv (IL)

(73) Assignee: TUDU HOLDINGS LTD., Tel-Aviv (IL)

(21) Appl. No.: 17/273,359

(22) PCT Filed: Jun. 27, 2019
(86) PCT No.: PCT/IL2019/050717

§ 371 Date: Mar. 4, 2021

(30) Foreign Application Priority Data
Sep. 6, 2018 (IL) 261670

(51) Int. Cl. A61K 33/06; A61K 31/205; A61K 47/22; A61K 9/08; A61K 9/00; A23L 33/10; A61K 9/48; A61P 1/16; A61P 3/10

(52) U.S. Cl. CPC A61K 33/06; A61K 31/205; A61K 47/22; A61K 9/08; A61P 3/10; A23L 33/10; A61K 9/48; A61P 1/16; A61K 9/0053

(57) ABSTRACT

The present invention, in some embodiments thereof, relates to nutrition and, more particularly, to magnesium and L-Carnitine containing formulations which enhance the effect of exercise on fat metabolism, and which are useful, for example, in the treatment of liver conditions, such as fatty liver disease, chronic liver disease, non-alcoholic fatty liver disease (NAFLD), alcoholic liver disease (ALD), hepatitis, fibrosis, cirrhosis and non-alcoholic steatohepatitis (NASH).

### FIELD OF THE INVENTION

The present invention, in some embodiments thereof, relates to nutrition and, more particularly, to magnesium and L-Carnitine containing formulations which enhance the effect of exercise on fat metabolism, and which are useful, for example, in the treatment of liver conditions, such as fatty liver disease, chronic liver disease, non-alcoholic fatty liver disease (NAFLD), alcoholic liver disease (ALD), hepatitis, fibrosis, cirrhosis and non-alcoholic steatohepatitis (NASH).

### BACKGROUND OF THE INVENTION

Magnesium is a natural element widely diffused in living organisms, especially in mammals, wherein the largest concentration thereof occurs in bones. A magnesium deficiency, or hypomagnesaemia, is often associated with increased incidence of diabetes mellitus, metabolic syndrome, and mortality rate from coronary artery disease (CAD). Magnesium deficiency in a living organism has also been associated with abnormal muscle excitability as well as convulsions, psychiatric disturbances, and calcium and/or potassium abnormalities.

The current daily Recommended Dietary Allowances for magnesium is 420 mg/day for males and 320 mg/day for females above 31 years, with an additional 300 mg/day during pregnancy or physical growth. Surveys show that a substantial number of adults in the United States fail to consume recommended daily amounts of magnesium.

Carnitine (vitamin Bt; 3-hydroxy-4-trimethylammonio-butanoate) is a quaternary ammonium compound biosynthesized from the amino acids lysine and methionine. In living cells, it is required for the transport of fatty acids from the cytosol into the mitochondria during the breakdown of lipids for the generation of metabolic energy.

Carnitine exists in two stereoisomers. The biologically active form is L-carnitine, whilst its enantiomer, D-carnitine, is biologically inactive.

L-carnitine is for humans an essential co-factor for the transport of long-chain fatty acids across the inner mitochondrial membrane into the matrix, where they are broken down for energy production (β-oxidation).

L-carnitine-L-tartrate is the salt of L-carnitine base with tartaric acid with the formula (C₇H₁₆NO₃)₂.C₄H₄O₆. It is a crystalline powder, with a melting point of 169-175° C., consisting of approximately 68% L-carnitine and 32% L-tartaric acid. It has a molecular weight of 472.5 g/mol and CAS Registry Number 36687-82-8. Its chemical name is: 3-carboxy-2-hydroxy-N,N,N-trimethyl-(2R)-1-propanaminium, 2:1 salt with (2R,3R)-2,3-dihydroxybutanedioic acid.

Its structural formula is:

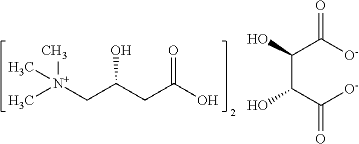

L-carnitine-containing products have been reported as improving exercise performance. These reports concern studies which demonstrated that L-carnitine plays an essential role in the body for producing energy from fat; ensuring athletic endurance; promoting recovery after exercise; providing the heart and immune cells with energy; and preventing early onset of fatigue during exercise.

Fatty liver disease is a reversible condition where large vacuoles of triglyceride fat accumulate in liver cells. The disease has multiple causes, but is associated primarily with excessive intake of alcohol or another drug or toxin, with obesity and insulin resistance, and with other conditions that influence fat metabolism. Non-alcoholic fatty liver disease (NAFLD) is one of the types of fatty liver disease, which occurs when fat is deposited (steatosis) in the liver due to causes other than excessive alcohol use. Non-alcoholic steatohepatitis (NASH) is the most extreme and fast progressing subtype of NAFLD. Alcoholic liver disease (ALD) encompasses liver manifestations related to alcohol overconsumption, including fatty liver, alcoholic hepatitis and chronic hepatitis with liver fibrosis and cirrhosis. Hepatitis includes the inflammation of liver tissue, fibrosis includes the formation of excess fibrosis connective tissue in an organ, e.g., the liver, and cirrhosis is a condition in which the liver does not function properly due to long term damage characterized by the replacement of normal liver tissue by scar tissue.

### SUMMARY OF THE INVENTION

In a search for improved formulations for treating various liver conditions, wherein the effect of the formulations is enhanced upon exercising, a novel formulation, which combines a source of magnesium and a source of L-carnitine, was prepared.

According to an aspect of some embodiments of the invention, there is provided a formulation containing a source of magnesium, a source of L-carnitine and a physiologically acceptable carrier.

According to an aspect of some embodiments of the invention, there is provided a formulation containing a source of magnesium, a source of L-carnitine, dihydroquercetin (DHQ) and a physiologically acceptable carrier.

According to an aspect of some embodiments of the invention, there is provided a formulation comprising magnesium lactate, L-carnitine-L-tartrate and an aqueous solution.

According to an aspect of some embodiments of the invention, there is provided a kit comprising packaging material and a source of magnesium, a source of L-carnitine and a physiologically acceptable carrier packaged within the packaging material, the kit indicated for the treatment liver conditions, such as fatty liver disease, chronic liver disease, non-alcoholic fatty liver disease (NAFLD), alcoholic liver disease (ALD), hepatitis, fibrosis, cirrhosis and non-alcoholic steatohepatitis (NASH).

According to an aspect of some embodiments of the invention, there is provided a kit comprising a packaging material and a formulation described herein packaged within the packaging material, the kit indicated for the treatment of liver conditions, such as fatty liver disease, chronic liver disease, non-alcoholic fatty liver disease (NAFLD), alcoholic liver disease (ALD), hepatitis, fibrosis, cirrhosis and non-alcoholic steatohepatitis (NASH).

According to an aspect of some embodiments of the invention, there is provided a nutrition supplement comprising a formulation described herein.

According to an aspect of some embodiments of the invention, there is provided a use of a formulation described herein in the manufacture of a nutrition supplement.

According to some embodiments the formulation further comprises an antioxidant.

According to some embodiments, the antioxidant is dihydroquercetin (DHQ).

According to some embodiments, the source of magnesium comprises magnesium lactate.

According to some embodiments, the source of L-carnitine comprises L-carnitine tartrate.

According to some embodiments, the formulation is in a liquid form.

According to some embodiments, the carrier is an aqueous solution.

According to some embodiments, the formulation is formulated for oral administration.

According to some embodiments, the formulation is indicated for use in combination with exercise.

According to some embodiments, the formulation is administered from about 10 minutes to about 3 hours prior to performing exercise.

According to some embodiments, the formulation is administered about one hour prior to performing exercise.

According to some embodiments, the formulation is administered once a day.

According to some embodiments, the formulation is administered for a time period that ranges from about 10 days to about 200 days.

According to some embodiments, the source of magnesium and the source of L-carnitine are packaged individually within the packaging material.

According to some embodiments, each of the source of magnesium, the source of L-carnitine and the carrier are packaged individually within the packaging material.

According to some embodiments, the source of magnesium and the carrier are packaged together within the packaging material, and the kit further comprises instruction to mix the source of L-carnitine with the source of magnesium and the carrier.

According to some embodiments, the kit further comprises an antioxidant packaged within the packaging material.

According to some embodiments, the antioxidant is dihydroquercetin (DHQ).

According to some embodiments, the kit is indicated for use in combination with exercise.

According to some embodiments, the kit is indicated for treating liver conditions, such as fatty liver disease, chronic liver disease, non-alcoholic fatty liver disease (NAFLD), alcoholic liver disease (ALD), hepatitis, fibrosis, cirrhosis and non-alcoholic steatohepatitis (NASH).

According to some embodiments, the nutrition supplement is indicated for use in combination with exercise.

According to some embodiments, the nutrition supplement is indicated for the treatment of liver conditions, such as fatty liver disease, chronic liver disease, non-alcoholic fatty liver disease (NAFLD), alcoholic liver disease (ALD), hepatitis, fibrosis, cirrhosis and non-alcoholic steatohepatitis (NASH).

Some embodiments of the invention are directed to a method of treating or reducing the severity of the chronic liver disease, fatty liver disease, non alcoholic fatty liver disease (NAFLD), alcoholic liver disease (ALD), hepatitis, fibrosis, cirrhosis or NASH comprising administering the formulation of the invention to a subject in need thereof. According to some embodiments, according to some embodiments, the formulation is administered prior to exercising.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF DETAILED EMBODIMENTS OF THE INVENTION

The present invention, in some embodiments thereof, relates to nutrition and, more particularly, but not exclusively, to magnesium and L-carnitine containing formulations, which are useful, for example, for treating liver conditions, such as fatty liver disease, chronic liver disease, non-alcoholic fatty liver disease (NAFLD), alcoholic liver disease (ALD), hepatitis, fibrosis, cirrhosis and non-alcoholic steatohepatitis (NASH).

It is to be understood that the invention is not necessarily limited in its application to the details set forth in the following description or exemplified by the examples. The invention may include any other embodiments and/or be practiced or carried out in various ways, whether described herein or not.

According to an aspect of some embodiments of the invention, there is provided a formulation containing a source or magnesium, a source of L-carnitine, and a physiologically acceptable carrier. Herein, “magnesium” refers to the magnesium ion Mg²⁺, except where indicated otherwise.

Hereinafter, the term “physiologically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

In some embodiments, the formulation further comprises an antioxidant.

In some embodiments, the antioxidant comprises a flavonoid. In some embodiments, the flavonoid comprises a flavanonol, that is, a flavonoid comprising a 2,3-dihydroflavonol backbone. Examples of antioxidant flavanonols include, for example, dihydroquercetin (DHQ) and dihydrokaempferol. DHQ is an exemplary antioxidant.

The antioxidant may be a chiral or achiral compound. In some embodiments wherein an antioxidant is chiral (e.g., DHQ), the compound may comprise any of the chiral forms of the compound, or any mixture thereof (e.g., a racemate). For example, DHQ may comprise (2R,3R)-DHQ, (2S,3S)-DHQ, (2R,3S)-DHQ, and/or (2S,3R)-DHQ. In some embodiments, the DHQ is (2R,3R)-DHQ.

Hence, according to some embodiments of the present invention, there is provided a formulation containing a source or magnesium, a source of L-carnitine, an antioxidant and a physiologically acceptable carrier.

According to some embodiments of the present invention, there is provided a formulation containing a source or magnesium, a source of L-carnitine, a flavanonol antioxidant and a physiologically acceptable carrier.

According to some embodiments of the present invention, there is provided a formulation containing a source of magnesium, a source of L-carnitine, dihydroquercetin (DHQ) (e.g., (2R,3R)-DHQ) and a physiologically acceptable carrier.

Herein, the phrase “source of magnesium” encompasses compounds, salts, complexes and compositions that generate magnesium (e.g., magnesium ions) in a physical medium and include, for example, salts and complexes of magnesium and mixtures (e.g., solutions, suspensions) comprising magnesium (e.g., magnesium ions).

In some embodiments, the source of magnesium is a pharmaceutically acceptable salt of magnesium.

As generally used herein, the phrase “pharmaceutically acceptable salt” refers to a charged species of the parent compound (e.g., Mg²⁺, L-carnitine) and its counter ion(s). The counter ion(s) is typically selected to modify the solubility characteristics of the parent compound and/or to reduce any significant irritation to an organism by the salt, while not abrogating the biological activity and properties of the administered compound. An example, without limitation, of a counter ion (e.g., anion) for a cation (e.g., magnesium, L-carnitine) in a pharmaceutically acceptable salt would be a carboxylate anion such as, but not limited to, acetate and lactate (e.g., as univalent anions), and tartrate (e.g., as a divalent anion), and the like. Examples, without limitation, of a counter ion (e.g., cation) for an anion in a pharmaceutically acceptable salt include ammonium, sodium and potassium.

A counter ion for a divalent ion such as magnesium (Mg²⁺) may comprise, for example, one or more univalent anions, which may be combined with magnesium at a 2:1 anion:magnesium molar ratio; one or more divalent anions, which may be combined with magnesium at a 1:1 anion:magnesium molar ratio; and a trivalent anion, which may be combined with magnesium at a 2:3 anion:magnesium molar ratio. In addition, combinations of univalent, divalent and trivalent anions may be used.

In some embodiments, the counter ion for magnesium consists of a univalent anion (one or more univalent anions) and/or a divalent anion (one or more divalent anions).

In some embodiments, the counter ion for magnesium is a univalent anion (one or more univalent anions).

Without being bound by any particular theory, it is believed that a magnesium salt with anions characterized by a relatively low valence (i.e., divalent and univalent anions), and especially univalent anions, will facilitate dissolution of the magnesium, and thereby enhance, for example, bioavailability of the magnesium, ease of formulation, and/or ease of administration of the formulation.

In some embodiments, the salt of magnesium comprises magnesium lactate. In some embodiments, the lactate comprises L-lactate. In some embodiments, the lactate comprises D-lactate. In some embodiments, the lactate comprises racemic lactate.

Without being bound by any particular theory, it is believed that magnesium lactate is a highly suitable source of magnesium, because it is a highly water-soluble salt, and because the lactate can further serve as a significant source of energy to the body when administered prior to performing exercise.

In some embodiments, the formulation comprises magnesium lactate (e.g., magnesium ions and lactic acid) as a source of magnesium.

Herein, the phrase “source of L-carnitine” encompasses pure L-carnitine, as well as salts, complexes, compounds or compositions that generate L-carnitine in a physiological medium, and mixtures (e.g., solutions, suspensions) comprising L-carnitine.

In some embodiments, the source of L-carnitine comprises zwitterionic L-carnitine, that is, L-carnitine comprising a positive charge (e.g., an ammonium cation) and an equal negative charge (e.g., carboxylate anion).

In some embodiments, the source comprises pure (e.g., zwitterionic) L-carnitine.

In some embodiments, the source comprises a pharmaceutically acceptable salt of L-carnitine.

In some embodiments, the pharmaceutically acceptable salt of L-carnitine comprises zwitterionic L-carnitine, a positively charged counter ion (e.g., cations described herein), and a negatively charged counter ion (e.g., anions described herein).

In some embodiments, the pharmaceutically acceptable salt of L-carnitine comprises positively charged L-carnitine (e.g., comprising a cationic ammonium group and a neutral carboxylic acid group) and one or more negatively charged counter ions (e.g., anions described herein).

A counter ion for positively charged L-carnitine may comprise, for example, one or more univalent anions, which may be combined with L-carnitine at a 1:1 anion:L-carnitine molar ratio; one or more divalent anions, which may be combined with L-carnitine at a 1:2 anion:L-carnitine molar ratio; and one or more trivalent anions, which may be combined with L-carnitine at a 1:3 anion:L-carnitine molar ratio. In addition, combinations of univalent, divalent and trivalent anions may be used.

In some embodiments, the counter ion for L-carnitine consists of a univalent anion (one or more univalent anions) and/or a divalent anion (one or more divalent anions).

Tartrate is an exemplary divalent anion.

In some embodiments, the tartrate comprises D-tartrate. In some embodiments, the tartrate comprises meso-tartrate. In some embodiments, the tartrate comprises racemic tartrate (L-tartrate and D-tartrate). In exemplary embodiments, the tartrate comprises L-tartrate.

In some embodiments, the counter ion for L-carnitine is a univalent anion (one or more univalent anions).

Without being bound by any particular theory, it is believed that an L-carnitine salt with anions characterized by a relatively low valence (i.e., divalent and univalent anions), may help to avoid interfering with dissolution of magnesium, when the L-carnitine salt is contacted with the magnesium.

The counter ions of magnesium and/or L-carnitine may be chiral or achiral. In some embodiments, when a compound or ion is chiral (e.g., lactate, tartrate), the compound may comprise any of the chiral forms of the compound or ion, or any mixture thereof (e.g., a racemate).

In some embodiments, the formulation is in liquid form, for example, a formulation wherein the physiologically acceptable carrier is a liquid. In exemplary embodiments, the physiologically acceptable carrier is an aqueous solution.

Herein, the term “aqueous solution” encompasses pure water (to which the sources of magnesium and L-carnitine are added) as well as water with solutes (e.g., solutes other than magnesium and L-carnitine).

In some embodiments, an aqueous solution contains both the carrier (e.g., water) and at least one other component of the formulation (e.g., magnesium, L-carnitine, or antioxidant). Thus, in such embodiments, the aqueous solution may be the source of magnesium or source of L-carnitine described herein.

An example of such a solution is magnesium-enriched water, which may optionally serve as both carrier and source of magnesium. Magnesium-enriched water suitable for use in the formulation may be prepared, for example, as described in International Patent Application PCT/IL2010/000843 (published as WO 2011/045795).

In some embodiments, a concentration of magnesium in a formulation in liquid form (e.g., as described herein) is in a range of from about 120 mg/l to about 3000 mg/l. In some embodiments, the concentration of magnesium is in a range of from about 200 mg/l to about 2000 mg/l. In some embodiments, the concentration of magnesium is in a range of from about 300 mg/l to about 1200 mg/l. In some embodiments, the concentration of magnesium is in a range of from about 400 mg/l to 800 mg/l. In exemplary embodiments, the concentration of magnesium is about 600 mg/l. In some embodiments, the concentration of magnesium is in a range of from about 120 mg/l to about 300 mg/l. In some embodiments, the concentration of magnesium is in a range of from about 300 mg/l to about 600 mg/l. In some embodiments, the concentration of magnesium is in a range of from about 600 mg/l to about 900 mg/l. In some embodiments, the concentration of magnesium is in a range of from about 900 mg/l to about 1200 mg/l. In some embodiments, the concentration of magnesium is in a range of from about 1200 mg/l to about 1500 mg/l. In some embodiments, the concentration of magnesium is in a range of from about 1500 mg/l to about 1800 mg/l. In some embodiments, the concentration of magnesium is in a range of from about 1800 mg/l to about 2000 mg/l.

In some embodiments, a concentration of L-carnitine in a formulation in liquid form (e.g., as described herein) is in a range of from about 1360 mg/l to about 10880 mg/l. In some embodiments, the concentration of L-carnitine is in a range of from about 2720 mg/l to about 10880 mg/l. In some embodiments, the concentration of L-carnitine is in a range of from about 2720 mg/l to about 8160 mg/l. In some embodiments, the concentration of L-carnitine is in a range of from about 4080 mg/l to about 6800 mg/l. In exemplary embodiments, the concentration of L-carnitine is about 5440 mg/l. In some embodiments, the concentration of L-carnitine is in a range of from about 1360 mg/l to about 2720 mg/l. In some embodiments, the concentration of L-carnitine is in a range of from about 2720 mg/l to about 4080 mg/l. In some embodiments, the concentration of L-carnitine is in a range of from about 4080 mg/l to about 5440 mg/l. In some embodiments, the concentration of L-carnitine is in a range of from about 5440 mg/l to about 6800 mg/l. In some embodiments, the concentration of L-carnitine is in a range of from about 6800 mg/l to about 8160 mg/l. In some embodiments, the concentration of L-carnitine is in a range of from about 8160 mg/l to about 9520 mg/l. In some embodiments, the concentration of L-carnitine is in a range of from about 9520 mg/l to about 10880 mg/l.

In some embodiments, a concentration of L-carnitine-L-tartrate in a formulation in liquid form (e.g., as described herein) is in a range of from about 2000 mg/l to about 16000 mg/l. In some embodiments, the concentration of L-carnitine-L-tartrate is in a range of from about 4000 mg/l to about 16000 mg/l. In some embodiments, the concentration of L-carnitine-L-tartrate is in a range of from about 4000 mg/l to about 12000 mg/l. In some embodiments, the concentration of L-carnitine-L-tartrate is in a range of from about 6000 mg/l to about 10000 mg/l. In exemplary embodiments, the concentration of L-carnitine-L-tartrate is about 8000 mg/l. In some embodiments, the concentration of L-carnitine-L-tartrate is in a range of from about 2000 mg/l to about 4000 mg/l. In some embodiments, the concentration of L-carnitine-L-tartrate is in a range of from about 4000 mg/l to about 6000 mg/l. In some embodiments, the concentration of L-carnitine-L-tartrate is in a range of from about 6000 mg/l to about 8000 mg/l. In some embodiments, the concentration of L-carnitine-L-tartrate is in a range of from about 8000 mg/l to about 10000 mg/l. In some embodiments, the concentration of L-carnitine-L-tartrate is in a range of from about 10000 mg/l to about 12000 mg/l.

One of ordinary skill in the art will be readily capable of selecting an amount of a source of magnesium and/or a source of L-carnitine which will provide suitable amount per dose/serving of magnesium and L-carnitine, as described herein.

According to some embodiments, the source of magnesium in the formulation comprises about 150 mg/l magnesium. According to some embodiments, the source of magnesium in the formulation comprises about 100-110 mg/l of magnesium. According to some embodiments, the source of magnesium in the formulation comprises about 110-120 mg/l of magnesium. According to some embodiments, the source of magnesium in the formulation comprises about 120-130 mg/l of magnesium. According to some embodiments, the source of magnesium in the formulation comprises about 130-140 mg/l of magnesium. According to some embodiments, the source of magnesium in the formulation comprises about 140-150 mg/l of magnesium. According to some embodiments, the source of magnesium in the formulation comprises about 150-160 mg/l of magnesium. According to some embodiments, the source of magnesium in the formulation comprises about 160-170 mg/l of magnesium. According to some embodiments, the source of magnesium in the formulation comprises about 170-180 mg/l of magnesium. According to some embodiments, the source of magnesium in the formulation comprises about 180-190 mg/l of magnesium. According to some embodiments, the source of magnesium in the formulation comprises about 190-200 mg/l of magnesium.

According to some embodiments, about 10% w/w of the magnesium source is magnesium. According to some embodiments, between about 5-15% w/w of the magnesium source is magnesium.

According to some embodiments, the magnesium source is magnesium lactate or magnesium citrate.

According to some embodiments, the amount of the magnesium source per dose of formulation is about 1500 mg. According to some embodiments, the amount of the magnesium source per dose of formulation is between about 1000-1250 mg. According to some embodiments, the amount of the magnesium source per dose of formulation is between about 1250-1500 mg. According to some embodiments, the amount of the magnesium source per dose of formulation is between about 1500-1750 mg. According to some embodiments, the amount of the magnesium source per dose of formulation is between about 1750-2000 mg.

According to some embodiments, the amount of the magnesium source per dose of formulation is between about 3-1500 mg. According to some embodiments, the amount of the magnesium source per dose of formulation is between about 3-10 mg. According to some embodiments, the amount of the magnesium source per dose of formulation is between about 10-50 mg. According to some embodiments, the amount of the magnesium source per dose of formulation is between about 50-100 mg. According to some embodiments, the amount of the magnesium source per dose of formulation is between about 100-200 mg. According to some embodiments, the amount of the magnesium source per dose of formulation is between about 200-300 mg. According to some embodiments, the amount of the magnesium source per dose of formulation is between about 300-400 mg. According to some embodiments, the amount of the magnesium source per dose of formulation is between about 400-500 mg. According to some embodiments, the amount of the magnesium source per dose of formulation is between about 500-600 mg. According to some embodiments, the amount of the magnesium source per dose of formulation is between about 600-700 mg. According to some embodiments, the amount of the magnesium source per dose of formulation is between about 700-800 mg. According to some embodiments, the amount of the magnesium source per dose of formulation is between about 800-900 mg. According to some embodiments, the amount of the magnesium source per dose of formulation is between about 900-1000 mg. According to some embodiments, the amount of the magnesium source per dose of formulation is between about 1000-1100 mg. According to some embodiments, the amount of the magnesium source per dose of formulation is between about 1100-1200 mg. According to some embodiments, the amount of the magnesium source per dose of formulation is between about 1200-1300 mg. According to some embodiments, the amount of the magnesium source per dose of formulation is between about 1300-1400 mg. According to some embodiments, the amount of the magnesium source per dose of formulation is between about 1400-1500 mg.

According to some embodiments, the amount of the L-carnitine source per dose of formulation is between about 100-2500 mg. According to some embodiments, the amount of the L-carnitine source per dose of formulation is between about 100-200 mg. According to some embodiments, the amount of the L-carnitine source per dose of formulation is between about 200-300 mg. According to some embodiments, the amount of the L-carnitine source per dose of formulation is between about 300-400 mg. According to some embodiments, the amount of the L-carnitine source per dose of formulation is between about 400-500 mg. According to some embodiments, the amount of the L-carnitine source per dose of formulation is between about 500-750 mg. According to some embodiments, the amount of the L-carnitine source per dose of formulation is between about 750-1000 mg. According to some embodiments, the amount of the L-carnitine source per dose of formulation is between about 1000-1250 mg. According to some embodiments, the amount of the L-carnitine source per dose of formulation is between about 1250-1500 mg. According to some embodiments, the amount of the L-carnitine source per dose of formulation is between about 1500-1750 mg. According to some embodiments, the amount of the L-carnitine source per dose of formulation is between about 1750-2000 mg. According to some embodiments, the amount of the L-carnitine source per dose of formulation is between about 2000-2250 mg. According to some embodiments, the amount of the L-carnitine source per dose of formulation is between about 2250-2500 mg.

According to some embodiments, the amount of the magnesium source per dose of formulation is about 1500 mg and the amount of free magnesium per dose of formulation is about 150 mg. According to some embodiments, the amount of the magnesium source per dose of formulation is between about 1000-1250 mg and the amount of the free magnesium is per dose of formulation between about 100-125 mg. According to some embodiments, the amount of the magnesium source per dose of formulation is between about 1250-1500 mg and the amount of the free magnesium per dose of formulation is between about 125-150 mg. According to some embodiments, the amount of the magnesium source per dose of formulation is between about 1500-1750 mg and the amount of the free magnesium per dose of formulation is between about 150-175 mg. According to some embodiments, the amount of the magnesium source per dose of formulation is between about 1750-2000 mg and the amount of the free magnesium per dose of formulation is between about 175-200 mg.

According to some embodiments, the amount of the magnesium source in the formulation is about 1500 mg, the amount of free magnesium is about 150 mg and the amount of the L-carnitine source is between about 1500-2000 mg.

In some embodiments, a concentration of DHQ in a formulation in liquid form (e.g., as described herein) is in a range of from about 20 mg/l to about 600 mg/l. In some embodiments, the concentration of DHQ is in a range of from about 40 mg/l to about 300 mg/l. In some embodiments, the concentration of DHQ is in a range of from about 60 mg/l to about 200 mg/l. In exemplary embodiments, the concentration of DHQ is about 60 mg/l. In some embodiments, the concentration of DHQ is in a range of from about 20 mg/l to about 40 mg/l. In some embodiments, the concentration of DHQ is in a range of from about 40 mg/l to about 60 mg/l. In some embodiments, the concentration of DHQ is in a range of from about 60 mg/l to about 80 mg/l. In some embodiments, the concentration of DHQ is in a range of from about 80 mg/l to about 100 mg/l. In some embodiments, the concentration of DHQ is in a range of from about 100 mg/l to about 120 mg/l. In some embodiments, the concentration of DHQ is in a range of from about 120 mg/l to about 140 mg/l. In some embodiments, the concentration of DHQ is in a range of from about 140 mg/l to about 160 mg/l. In some embodiments, the concentration of DHQ is in a range of from about 160 mg/l to about 180 mg/l. In some embodiments, the concentration of DHQ is in a range of from about 180 mg/l to about 200 mg/l. In some embodiments, the concentration of DHQ is in a range of from about 200 mg/l to about 220 mg/l. In some embodiments, the concentration of DHQ is in a range of from about 220 mg/l to about 240 mg/l. In some embodiments, the concentration of DHQ is in a range of from about 240 mg/l to about 260 mg/l. In some embodiments, the concentration of DHQ is in a range of from about 260 mg/l to about 280 mg/l. In some embodiments, the concentration of DHQ is in a range of from about 280 mg/l to about 300 mg/l.

An exemplary formulation according to some embodiments of the present invention is presented in Example 1 hereinafter.

Thus, a general formulation, according to some embodiments of the present invention, comprises a source of magnesium, e.g., releasing about 150 mg magnesium, a source of L-carnitine, e.g., containing about 2,000 mg of the source of L-carnitine, water, and optionally dihydroquercetin (DHQ), e.g., at an amount of 15-50 mg.

An exemplary formulation contains 1,500 mg magnesium lactate (approximately 150 mg magnesium and 1,350 mg lactic acid); 2000 mg L-carnitine-L-tartrate (approximately 1360 mg L-carnitine); 15 mg DHQ, and water to complete a 250 ml formulation.

Thus, a general formulation, according to some embodiments of the present invention, comprises a source of magnesium, e.g., releasing about 150 mg magnesium, a source of L-carnitine, e.g., containing about 1500-2000 mg of the source of L-carnitine, water, and optionally dihydroquercetin (DHQ), e.g., at an amount of 15-50 mg.

Other formulations may contain other sources of magnesium (e.g., in a form of magnesium-enriched water); other sources of L-carnitine (e.g., as a pure crystalline form of free base L-carnitine, a HCl salt of L-carnitine; and more); other amounts of the source of magnesium; other amounts of a source of L-carnitine; other anti-oxidants; and other amounts, including absence, of DHQ or any other antioxidant.

The formulation described herein may be provided for use in a form of a nutrition supplement.

Hence, according to another aspect of embodiments of the invention, there is provided a use of the formulation described herein in the manufacture of a nutrition supplement. In some embodiments, the nutrition supplement is for use in combination with exercise (e.g., as described herein).

According to an aspect of some embodiments of the present invention there is provided a nutrition supplement which comprises a formulation as described herein. In some embodiments, the nutrition supplement is for use in combination with exercise (e.g., as described herein).

In some embodiments, the nutrition supplement includes one or more additional nutrients, in addition to the magnesium, L-carnitine and optional antioxidant as described herein. Examples of suitable nutrients include, without limitation, essential minerals (e.g., calcium, iron, potassium, sodium), vitamins (e.g., vitamins A, B₁, B₂, B₃, B₆, B₁₂, C, D, E and/or K), and amino acids (e.g., any of the 20 standard amino acids, any of the 9 essential amino acids).

In some embodiments, the nutrition supplement comprises additional additives and/or active ingredients which may provide a beneficial effect. Examples of such additives include, but are not limited to, caffeine, L-arginine, ornithine, guarana, ginseng, and ginkgo balboa.

Any of the formulations and nutrition supplements described herein are useful for the treatment of liver conditions, as detailed herein, in a subject, upon exercising.

In some embodiments, the method is for treating liver conditions, such as fatty liver disease, chronic liver disease, non-alcoholic fatty liver disease (NAFLD), alcoholic liver disease (ALD), hepatitis, fibrosis, cirrhosis and non-alcoholic steatohepatitis (NASH) in a subject upon exercising.

In any of these embodiments, the method comprises administering the formulation and/or nutrition supplement as described herein to the subject prior to exercising.

Fatty liver disease encompasses both non-alcoholic fatty liver disease and fatty liver disease associated with alcoholic liver disease.

In some embodiments the fatty liver disease is non-alcoholic fatty liver disease.

In some embodiments the fatty liver disease is associated with alcoholic liver disease.

Fatty liver disease may be determined in accordance with known criteria used in the art of medicine, including for example, imaging of the liver (e.g., by ultrasonography, computed tomography, magnetic resonance imaging) and/or by tests for biomarkers (e.g., elevated liver enzymes). Examples of suitable non-invasive tests for fatty liver disease include, without limitation, the FibroTest and the SteatoTest.

In any of the aspects described herein, the formulation and/or nutrition supplement is optionally administered prior to beginning to perform exercise.

In some embodiments, the administration of the formulation and/or nutrition supplement is performed up to about 3 hours prior to exercising. In some embodiments, the administration of the formulation and/or nutrition supplement is performed from about 10 minutes to about 3 hours prior to exercising.

In some embodiments, the administration of the formulation and/or nutrition supplement is performed up to about 2 hours prior to exercising. In some embodiments, the administration of the formulation and/or nutrition supplement is performed from about 10 minutes to about 2 hours prior to exercising.

In some embodiments, the administration of the formulation and/or nutrition supplement is performed up to about 1.5 hours prior to exercising. In some embodiments, the administration of the formulation is performed from about 10 minutes to about 1.5 hours prior to exercising.

In some embodiments, the administration of the formulation and/or nutrition supplement is performed at least about 10 minutes prior to exercising. In some embodiments, the administration of the formulation and/or nutrition supplement is performed at least about 20 minutes prior to exercising. In some embodiments, the administration of the formulation and/or nutrition supplement is performed at least about 30 minutes prior to exercising. In some embodiments, the administration of the formulation and/or nutrition supplement is performed at least about 40 minutes prior to exercising. In some embodiments, the administration of the formulation and/or nutrition supplement is performed at least about one hour prior to exercising.

In some embodiments, the administration of the formulation and/or nutrition supplement is performed from about 20 minutes to about 2 hours prior to exercising. In some embodiments, the administration of the formulation and/or nutrition supplement is performed from about 30 minutes to about 1.5 hours prior to exercising. In some embodiments, the administration of the formulation and/or nutrition supplement is performed about one hour prior to exercising.

In some embodiments, the administration of the formulation and/or nutrition supplement is performed once a day. In some such embodiments, a daily exercise may be performed shortly thereafter, for example, within about three hours or less after administration of the formulation, e.g., as described herein. The daily exercise may be performed as a single exercise session or as a series of relatively short exercise sessions.

In some such embodiments, a daily exercise is completed within about 3 hours or less after the administration of the formulation and/or nutrition supplement.

In some embodiments, the administration of the formulation and/or nutrition supplement (and in some embodiments, the accompanying exercise) is performed over a time period of at least about 10 days. In some embodiments, the time period is at least about 14 days. In some embodiments, the time period is at least about 20 days. In some embodiments, the time period is at least about 28 days. In some embodiments, the time period is at least about one month. In some embodiments, the time period is at least about two months. In some embodiments, the time period is at least about 3 months. In some embodiments, the time period is at least exercising 4 months. In some embodiments, the time period is at least exercising 5 months. In some embodiments, the time period is at least exercising 6 months.

In some embodiments, the administration of the formulation and/or nutrition supplement (and in some embodiments, the accompanying exercise) is performed over a time period of up to about 365 days. In some embodiments, the time period is up to about 200 days. In some embodiments, the time period is up to about 150 days. In some embodiments, the time period is up to about 100 days. In some embodiments, the time period is up to about 60 days. In some embodiments, the time period is up to about 30 days.

In some embodiments, the administration of the formulation and/or nutrition supplement (and in some embodiments, the accompanying exercise) is performed over a time period of from about 10 days to about 365 days. In some embodiments, the time period is from about 10 days to about 200 days. In some embodiments, the time period is from about 14 days to about 150 days. In some embodiments, the time period is from about 20 days to about 100 days. In some embodiments, the time period is from about 28 days to about 60 days. In some embodiments, the time period is from about one day to about 200 days. In some embodiments, the time period is from about one to 10 days. In some embodiments, the time period is from about 10 to 20 days. In some embodiments, the time period is from about 20 to 50 days. In some embodiments, the time period is from about 50 to 100 days. In some embodiments, the time period is from about 100 to 150 days. In some embodiments, the time period is from about 150 to 200 days. In some embodiments, the time period is from about 200 to 250 days. In some embodiments, the time period is from about 250 to 300 days. In some embodiments, the time period is from about 300 to 365 days.

In exemplary embodiments, the administration of the formulation and/or nutrition supplement (and in some embodiments, the accompanying exercise) is performed over a time period of about 28 days.

It is believed that due to the efficacy of the formulation described herein, a regimen comprising the administration of the formulation and exercising will be more effective in treating liver conditions, such as fatty liver disease, chronic liver disease, non-alcoholic fatty liver disease (NAFLD), alcoholic liver disease (ALD), hepatitis, fibrosis, cirrhosis and non-alcoholic steatohepatitis (NASH), than a corresponding regimen of exercise alone or a corresponding regimen of exercise and the administration of L-carnitine. A “corresponding regimen” refers to a regimen with an equivalent amount of exercise and/or L-carnitine (e.g., in dose and time).

Characterization of the effect of a combination of exercise and a formulation and/or nutrition supplement described herein may be performed by studying a group of subjects and averaging the results, as commonly performed in the art. Similarly, comparisons between the effects of a combination of exercise and a formulation described herein and exercise, alone or a combination of exercise and L-carnitine (as described herein), may be characterized by comparing average results for groups of subjects. In some embodiments, each group comprises at least 4 subjects. In some embodiments, each group comprises at least 5 subjects. In some embodiments, each group comprises at least 6 subjects. In some embodiments, each group comprises at least 8 subjects. In some embodiments, each group comprises at least 10 subjects. In some embodiments, each group comprises at least 15 subjects. In some embodiments, each group comprises at least 20 subjects.

In some embodiments of any of the aspects described herein, the amount of magnesium in the formulation to be administered in per dose (e.g., a daily administration) is in a range of from about 30 mg to about 750 mg. In some embodiments, the amount of magnesium is in a range of from about 50 mg to about 500 mg. In some embodiments, the amount of magnesium is in a range of from about 75 mg to about 300 mg. In some embodiments, the amount of magnesium is in a range of from about 100 mg to about 200 mg. In exemplary embodiments, the amount of magnesium is about 150 mg. In some embodiments, the amount of magnesium is in a range of from about 30 mg to about 50 mg. In some embodiments, the amount of magnesium is in a range of from about 50 mg to about 75 mg. In some embodiments, the amount of magnesium is in a range of from about 75 mg to about 100 mg. In some embodiments, the amount of magnesium is in a range of from about 100 mg to about 125 mg. In some embodiments, the amount of magnesium is in a range of from about 125 mg to about 150 mg. In some embodiments, the amount of magnesium is in a range of from about 150 mg to about 175 mg. In some embodiments, the amount of magnesium is in a range of from about 175 mg to about 200 mg. In some embodiments, the amount of magnesium is in a range of from about 200 mg to about 250 mg. In some embodiments, the amount of magnesium is in a range of from about 250 mg to about 300 mg. In some embodiments, the amount of magnesium is in a range of from about 300 mg to about 350 mg. In some embodiments, the amount of magnesium is in a range of from about 350 mg to about 400 mg. In some embodiments, the amount of magnesium is in a range of from about 400 mg to about 450 mg. In some embodiments, the amount of magnesium is in a range of from about 450 mg to about 500 mg.

In some embodiments of any of the aspects described herein, the amount of L-carnitine in the formulation to be administered per dose (e.g., a daily administration) is in a range of from about 340 mg to about 2720 mg. In some embodiments, the amount of L-carnitine is in a range of from about 680 mg to about 2720 mg. In some embodiments, the amount of L-carnitine is in a range of from about 680 mg to about 2040 mg. In some embodiments, the amount of L-carnitine is in a range of from about 1020 mg to about 1700 mg. In exemplary embodiments, the amount of L-carnitine is about 1360 mg. In some embodiments, the amount of L-carnitine is in a range of from about 340 mg to about 680 mg. In some embodiments, the amount of L-carnitine is in a range of from about 680 mg to about 1020 mg. In some embodiments, the amount of L-carnitine is in a range of from about 1020 mg to about 1360 mg. In some embodiments, the amount of L-carnitine is in a range of from about 1360 mg to about 1700 mg. In some embodiments, the amount of L-carnitine is in a range of from about 1700 mg to about 2040 mg. In some embodiments, the amount of L-carnitine is in a range of from about 2040 mg to about 2380 mg. In some embodiments, the amount of L-carnitine is in a range of from about 2380 mg to about 2720 mg.

In some embodiments of any of the aspects described herein, the L-carnitine is in the form of L-carnitine-L-tartarte, and the amount of L-carnitine-L-tartrate in the formulation to be administered per dose (e.g., a daily administration) is in a range of from about 500 mg to about 4000 mg. In some embodiments, the amount of L-carnitine-L-tartrate is in a range of from about 1000 mg to about 4000 mg. In some embodiments, the amount of L-carnitine-L-tartrate is in a range of from about 1000 mg to about 3000 mg. In some embodiments, the amount of L-carnitine-L-tartrate is in a range of from about 1500 mg to about 2500 mg. In exemplary embodiments, the amount of L-carnitine-L-tartrate is about 2000 mg. In some embodiments, the amount of L-carnitine-L-tartrate is in a range of from about 500 mg to about 750 mg. In some embodiments, the amount of L-carnitine-L-tartrate is in a range of from about 750 mg to about 1000 mg. In some embodiments, the amount of L-carnitine-L-tartrate is in a range of from about 1000 mg to about 1250 mg. In some embodiments, the amount of L-carnitine-L-tartrate is in a range of from about 1250 mg to about 1500 mg. In some embodiments, the amount of L-carnitine-L-tartrate is in a range of from about 1500 mg to about 1750 mg. In some embodiments, the amount of L-carnitine-L-tartrate is in a range of from about 1750 mg to about 2000 mg. In some embodiments, the amount of L-carnitine-L-tartrate is in a range of from about 2000 mg to about 2250 mg. In some embodiments, the amount of L-carnitine-L-tartrate is in a range of from about 2250 mg to about 2500 mg. In some embodiments, the amount of L-carnitine-L-tartrate is in a range of from about 2500 mg to about 2750 mg. In some embodiments, the amount of L-carnitine-L-tartrate is in a range of from about 2750 mg to about 3000 mg.

In some embodiments of any of the aspects described herein, the amount of DHQ in the formulation to be administered per dose (e.g., a daily administration) is in a range of from about 5 mg to about 150 mg. In some embodiments, the amount of DHQ is in a range of from about 10 mg to about 75 mg. In some embodiments, the amount of DHQ is in a range of from about 15 mg to about 50 mg. In exemplary embodiments, the amount of DHQ is about 15 mg. In some embodiments, the amount of DHQ is in a range of from about 10 mg to about 20 mg. In some embodiments, the amount of DHQ is in a range of from about 20 mg to about 30 mg. In some embodiments, the amount of DHQ is in a range of from about 30 mg to about 40 mg. In some embodiments, the amount of DHQ is in a range of from about 40 mg to about 50 mg. In some embodiments, the amount of DHQ is in a range of from about 50 mg to about 60 mg. In some embodiments, the amount of DHQ is in a range of from about 60 mg to about 70 mg. In some embodiments, the amount of DHQ is in a range of from about 70 mg to about 80 mg. In some embodiments, the amount of DHQ is in a range of from about 80 mg to about 90 mg. In some embodiments, the amount of DHQ is in a range of from about 90 mg to about 100 mg. In some embodiments, the amount of DHQ is in a range of from about 100 mg to about 110 mg. In some embodiments, the amount of DHQ is in a range of from about 110 mg to about 120 mg. In some embodiments, the amount of DHQ is in a range of from about 120 mg to about 130 mg. In some embodiments, the amount of DHQ is in a range of from about 130 mg to about 140 mg. In some embodiments, the amount of DHQ is in a range of from about 140 mg to about 150 mg.

An aqueous carrier may be beneficial prior to exercise by providing water required by the body during exercise.

Hence, in some embodiments of any of the aspects described herein, the volume of a liquid formulation based on an aqueous solution as described herein, which is to be administered in a single administration (e.g., a daily administration containing suitable amounts of magnesium and L-carnitine, and optionally DHQ, as described herein), contains a substantial amount of water, such that the liquid formulation has a substantial volume, for example, at least about 0.2 ml. In some embodiments, the volume of the formulation is in a range of from about 0.2 ml to about 1.0 ml. In some embodiments, the volume of the formulation is in a range of from about 1.0 ml to about 10 ml. In some embodiments, the volume of the formulation is in a range of from about 10 ml to about 25 ml. In some embodiments, the volume of the formulation is in a range of from about 25 ml to about 50 ml. In some embodiments, the volume of the formulation is in a range of from about 50 ml to about 100 ml. In some embodiments, the volume of the formulation is in a range of from about 50 ml to about 500 ml. In some embodiments, the volume is in a range of from about 100 ml to about 500 ml. In some embodiments, the volume is in a range of from about 150 ml to about 400 ml. In some embodiments, the volume is in a range of from about 200 ml to about 300 ml. In some embodiments, the volume is about 250 ml. In some embodiments, the volume is in a range of from about 50 ml to about 100 ml. In some embodiments, the volume is in a range of from about 100 ml to about 150 ml. In some embodiments, the volume is in a range of from about 150 ml to about 200 ml. In some embodiments, the volume is in a range of from about 200 ml to about 250 ml. In some embodiments, the volume is in a range of from about 250 ml to about 300 ml. In some embodiments, the volume is in a range of from about 300 ml to about 350 ml. In some embodiments, the volume is in a range of from about 350 ml to about 400 ml. In some embodiments, the volume is in a range of from about 400 ml to about 450 ml. In some embodiments, the volume is in a range of from about 450 ml to about 500 ml.

According to some embodiments, the formulation of the invention comprising palatinose, citric acid, carnitine L, magnesium citrate, flavorings, such as wild berry flavoring and cranberry flavoring, ascorbic acid, sucralose, potassium sorbate and water.

According to some embodiments, the formulation comprises about 1 gr/per dose palatinose, about 0.2 gr/per dose citric acid, about 1.5 gr/per dose carnitine L, about 1.5 gr/per dose magnesium citrate, about 0.1 gr/per dose wild berry flavoring, about 0.05 gr/per dose cranberry flavoring, about 0.01 gr/per dose ascorbic acid, about 0.012 gr/per dose sucralose, about 0.0225 gr/per dose potassium sorbate and about 10.6055 gr/per dose water, wherein the dose is of about 15 ml.

Techniques for formulation and administration of active agents may be found in “Remington's Pharmaceutical Sciences” Mack Publishing Co., Easton, Pa., latest edition.

Formulations of embodiments the present invention may be manufactured by processes well known in the art, e.g., by means of conventional mixing, dissolving, granulating, dragee-making, levigating, emulsifying, encapsulating, entrapping or lyophilizing processes.

Formulations in accordance with embodiments of the present invention thus may be formulated in conventional manner using one or more physiologically acceptable carriers comprising excipients and auxiliaries, which facilitate processing of the sources of magnesium and L-carnitine into preparations which can be used pharmaceutically. Proper formulation is dependent upon the route of administration chosen.

In some embodiments, the formulation is formulated for oral administration.

For oral administration, the magnesium and L-carnitine of embodiments the invention can be formulated readily by combining the sources of magnesium and L-carnitine with physiologically acceptable carriers well known in the art. Such carriers enable the magnesium and L-carnitine to be formulated as tablets, pills, dragees, capsules, liquids, gels, syrups, slurries, suspensions, and the like, for oral ingestion by a subject.

In exemplary embodiments, the formulation or nutrition supplement is formulated as a beverage, that is, a liquid which can be drunk by a typical subject in relatively large amounts (e.g., at least 50 ml) with no significant discomfort or adverse effects. Formulation of a beverage will generally include water or an aqueous solution as the carrier. The beverage may optionally include further ingredients known in the art for enhancing palatability of beverages, e.g., flavoring agents, sweeteners, coloring agents, and the like.

Formulations and nutrition supplements for oral use can be made using a solid excipient, optionally grinding the resulting mixture, and processing the mixture of granules, after adding suitable auxiliaries if desired, to obtain tablets or dragee cores. Suitable excipients include, in particular, fillers such as sugars, including lactose, sucrose, mannitol and/or sorbitol; cellulose preparations such as, for example, maize starch, wheat starch, rice starch, potato starch, gelatin, gum tragacanth, methylcellulose, hydroxypropylmethyl-cellulose, and/or sodium carboxymethylcellulose; and/or physiologically acceptable polymers such as polyvinylpyrrolidone (PVP). If desired, disintegrating agents may be added, such as cross-linked polyvinylpyrrolidone, agar, and/or alginic acid or a salt thereof such as sodium alginate.

Dragee cores may be provided with suitable coatings. For this purpose, concentrated sugar solutions may be used which may optionally contain gum arabic, talc, polyvinylpyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active magnesium and/or L-carnitine doses.

Formulations and nutrition supplements which can be used orally further include push-fit capsules prepared from gelatin, as well as soft, sealed capsules prepared from gelatin and a plasticizer, such as glycerol or sorbitol. The push-fit capsules may contain the active ingredients in admixture with fillers, such as lactose, binders, such as starches, lubricants, such as talc or magnesium stearate, and optionally stabilizers. In soft capsules, the magnesium and L-carnitine may be dissolved or suspended in suitable liquids, such as water, aqueous solutions, fatty oils, liquid paraffin, or liquid polyethylene glycols. In addition, stabilizers may be added.

All formulations for oral administration should be in dosages suitable for the chosen route of administration.

For injection, the magnesium and L-carnitine of embodiments of the invention may be formulated in aqueous solutions, such as physiologically compatible buffers such as Hank's solution, Ringer's solution, or physiological saline buffer with or without organic solvents such as propylene glycol, polyethylene glycol.

For transmucosal administration, penetrants may be used in the formulation.

For buccal administration, the compositions may take the form of tablets or lozenges formulated in conventional manner.

For administration by inhalation, the magnesium and L-carnitine for use according to the present invention may be delivered in the form of an aerosol spray presentation (which typically includes powdered, liquefied and/or gaseous carriers) from a pressurized pack or a nebulizer, with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the sources of magnesium and L-carnitine and a suitable powder base such as, but not limited to, lactose or starch.

The sources of magnesium and L-carnitine described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Formulations for parenteral administration include aqueous solutions of the sources of magnesium and L-carnitine in water-soluble form. Additionally, suspensions of the sources of magnesium and L-carnitine may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the magnesium and L-carnitine to allow for the preparation of highly concentrated solutions.

Alternatively, the sources of magnesium and L-carnitine may be in powder form for constitution with a suitable vehicle, e.g., water, before use.

The sources of magnesium and L-carnitine described herein may also be formulated in rectal compositions such as suppositories or retention enemas, using, e.g., conventional suppository bases such as cocoa butter or other glycerides.

The formulations and nutrition supplements described herein may also comprise suitable solid or gel phase carriers or excipients. Examples of such carriers or excipients include, but are not limited to, calcium carbonate, calcium phosphate, various sugars, starches, cellulose derivatives, gelatin and polymers, such as polyethylene glycols.

In some embodiments of any of the aspects described herein, the formulation or nutrition supplement is in a unit dosage form.

The term “unit dosage form”, as used herein, describes physically discrete units, each unit containing a predetermined quantity of active ingredients (e.g., magnesium and L-carnitine) calculated to produce the desired effect, in association with at least one physiologically acceptable carrier, diluent, excipient, or combination thereof.

In some embodiments, the unit dosage form comprises an amount of magnesium and L-carnitine (and optionally also an antioxidant such as DHQ) suitable for a formulation to be administered per dose (e.g., a daily administration), as described herein (e.g., from 30 mg to 750 mg magnesium, from 340 mg to 2720 mg L-carnitine (e.g., from 500 mg to 4000 mg L-carnitine-L-tartrate), e.g., from 5 mg to 150 mg DHQ).

Composition unit dosage forms for oral administration include sachets, pills, caplets, capsules, tablets, or discrete (e.g., separately packaged) units of powder, granules, or suspensions or solutions in aqueous or non-aqueous media.

In some embodiments, the unit dosage form comprises the formulation in a liquid form (e.g., suspension or solution), for example, a beverage (e.g., as described herein). The liquid of the unit dosage form may be packaged in a suitable packaging, for example, a bottle or a can.

In some embodiments of any of the aspects described herein, the volume of liquid formulation (or nutrition supplement) in a unit dosage form is at least about 50 ml. In some embodiments, the volume is in a range of from about 50 ml to about 500 ml. In some embodiments, the volume is in a range of from about 100 ml to about 500 ml. In some embodiments, the volume is in a range of from about 150 ml to about 400 ml. In some embodiments, the volume is in a range of from about 200 ml to about 300 ml. In some embodiments, the volume is about 250 ml.

In some embodiments, the unit dosage form is in a form of a solid (e.g., sachets, pills, caplets, capsules, tablets, or discrete units of powder, granules) or concentrated liquid (e.g., a syrup) and is prepared for use by mixing with a volume of water to form a suspension or solution. The volume of water may be determined so as to result in a liquid formulation described herein (e.g., having a volume of from about 50 ml to about 500 ml).

According to another aspect of embodiments of the invention, there is provided a kit comprising a packaging material, a source of magnesium (e.g., as described herein), a source of L-carnitine (e.g., as described herein) and a physiologically acceptable carrier (e.g., as described herein).

In some embodiments, the kit comprises two or more individually packaged components of a formulation described herein which may be combined by a user to form the formulation.

In some embodiments, the source of magnesium and the source of L-carnitine described herein are packaged individually (e.g., as separate components within the kit). In some embodiments, at least one of the aforementioned sources is packaged as a powder.

In some embodiments, a carrier as described herein is packaged individually (e.g., as a third component within the kit).

In some embodiments, the carrier is packaged in combination with another component, such as the source of magnesium or the source of L-carnitine, for example, in a form of a solution or suspension of the source of magnesium or source of L-carnitine.

In some embodiments, the source of magnesium and carrier are packaged in combination, for example, as a solution containing magnesium (e.g., magnesium-enriched water). In some embodiments, the L-carnitine is packaged separately as a solid (e.g., powder) which may be added to the carrier in combination with magnesium.

It is to be appreciated that magnesium is an element and thus resistant to degradation, whereas L-carnitine and many antioxidants (e.g., DHQ) are organic compounds, and therefore more susceptible to degradation, especially when in a liquid phase.

Hence, in some embodiments, a liquid carrier is packaged in combination with the source of magnesium, and organic compounds in the kit (e.g., L-carnitine, DHQ) are packaged individually in solid form (which is generally less susceptible than liquid forms to degradation), for example, as a powder. In some embodiments, the organic compounds are packaged individually. In some embodiments, the organic compounds are packaged in combination, e.g., as a powder mixture.

The active ingredients (e.g., magnesium and L-carnitine) according to any of the aspects of embodiments of the present invention may, if desired, be presented in a pack or dispenser device, such as an FDA (the U.S. Food and Drug Administration) approved kit, which may contain one or more unit dosage forms containing the active ingredients. The pack may, for example, comprise metal or plastic foil, such as, but not limited to, a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising formulations described herein may also be prepared, placed in an appropriate container, and labeled for the treatment of an indicated condition, as is detailed herein.

Thus, according to some embodiments of any of the aspects of embodiments of the present invention, the formulations, kits or nutrition supplements described herein are packaged in a packaging material and identified in print, in or on the packaging material, for any of the uses as described herein, including for use in the treatment of a condition selected from the group consisting of excess weight, fatty liver disease and chronic fatigue syndrome (as described herein). In some embodiments, the formulation, kit, or nutrition supplement is indicated for use in combination with exercise (e.g., as described herein). As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any numeral value (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures, either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which, together with the above descriptions, illustrate some embodiments of the invention in a non-limiting fashion.

### Example 1 (Prophetic)

Formulation:

A general formulation, according to some embodiments of the present invention, comprises a source of magnesium, e.g., releasing about 150 mg magnesium, a source of L-carnitine, e.g., containing about 2,000 mg of the source of L-carnitine, water, and optionally dihydroquercetin (DHQ), e.g., at an amount of 15-50 mg.

An exemplary formulation contains 1,500 mg magnesium lactate (approximately 150 mg magnesium and 1,350 mg lactic acid); 1360 mg L-Carnitine (Approximately 2000 mg L-carnitine-L-tartrate); 15 mg DHQ, and water to complete a 250 ml formulation. Other formulations may contain other sources of magnesium (e.g., in a form of magnesium-enriched water); other sources of L-carnitine (e.g., as a pure crystalline form of free base L-carnitine, a HCl salt of L-carnitine; and more); other amounts of the source of magnesium; other amounts of a source of L-carnitine; other anti-oxidants; and other amounts, including absence, of DHQ or any other antioxidant.

Study Protocol:

A clinical study will be conducted for demonstrating the effect of a combined formulation containing a source of magnesium and a source of L-carnitine.

100 volunteers suffering from liver conditions, such as fatty liver disease, chronic liver disease, non-alcoholic fatty liver disease (NAFLD), alcoholic liver disease (ALD), hepatitis, fibrosis, cirrhosis and non-alcoholic steatohepatitis (NASH), at an age of from 20 to 70, will be recruited for the study.

Each volunteer will be instructed to consume a 250 ml formulation about an hour before exercising, and to perform a one-hour exercise on a treadmill (walking at a speed of 6.5 kilometers per hour).

Volunteers will be divided into the following groups:

L-carnitine group—receiving a formulation containing two grams of L-carnitine;

Test group—receiving the abovementioned exemplary formulation containing a source of magnesium, a source of L-carnitine, and DHQ.

The treatment regimen (diet, exercise and formulations) will be applied for 4 weeks.

### Example 2 (Prophetic)

Patients with fatty liver disease (e.g., non-alcoholic fatty liver disease) will be divided into two groups (e.g., of 20 patients each). Patients in one group will be administered the formulation described in Example 1 (e.g., for 3 months), whereas patients in the other group will receive a placebo during the same time period. Administration of the formulation and placebo will be performed in conjunction with a suitable exercise regimen (e.g., as described in Example 1).

The effect of the treatment on the liver (relative to the placebo) will be assessed using a SteatoTest, which estimates steatosis, and will be performed in Rambam Laboratories in Jerusalem, Israel.

### Example 3

OBJECTIVES: To evaluate the effectiveness of the combination of a formulation comprising amino acid L-carnitine and magnesium together with exercise, when treating patients with NAFLD.

MATERIALS & METHODS: A Phase 3, randomized, double blind, placebo-controlled, NAFLD clinical trial was performed between October 2017 and May 2018, at the Liver Diseases Unit at Haemek Medical Center, Afula, Israel. Patients were randomly assigned and double blinded to two groups: The group was the “research group” which received a test formulation as detailed in Table 1 below for 16 weeks. The group was the “control group” which received a placebo (mineral water) for 8 weeks followed by an additional 8 weeks of the test formulation. Steatosis and liver fibrosis were assessed according to shear-wave elastography sonographic measurements using the supersonic sonographic unit (France) at the beginning and end of the study.

| | Amount (gr/per |
|---|---|
| Ingredients | dose of 15 ml) |
| Palatinose | 1 |
| Citric acid | 0.2 |
| Carnitine L | 1.5 |
| Magnesium citrate | 1.5 |
| Wild berry flavoring (70517/2 flavor) | 0.1 |
| Cranberry flavoring | 0.05 |
| Ascorbic acid (vitamin C) | 0.01 |
| Sucralose | 0.012 |
| Caffeine | 0 |
| Potassium sorbate | 0.0225 |
| Water | 10.6055 |

RESULTS: A total of 22 patients were recruited. The research group and the control group consisted of 11 patients each. In the research group, the aspartate aminotransferase (AST), alanine aminotransferase (ALT) and c-reactive protein (CRP) levels decreased significantly during the 16 weeks of treatment; however, the lipid profile and the insulin levels did not change significantly. In the control group there were no significant changes in AST, ALT and CRP levels and/or in the lipid profile or the insulin levels. Shear wave elastography failed to demonstrate a statistically significant difference between the two groups in terms of fibrosis.

CONCLUSIONS: Treatment consisting of L-carnitine and magnesium significantly improved liver transaminase levels in patients with NAFLD, especially levels of AST. There was no significant difference of fibrosis grade between the two groups.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All citation or identification of any reference in this application shall not be construed as an admission that such reference is available as prior art to the present invention. To the extent that section headings are used, they should not be construed as necessarily limiting.

What is claimed is:

1. A formulation containing a source of magnesium, a source of L-carnitine and a physiological acceptable carrier for use in treating a liver condition in combination with exercise.

2. The formulation of claim 1, further comprising an antioxidant.

3. The formulation of claim 2, wherein the antioxidant is a flavanonol antioxidant

4. The formulation of claim 3, wherein said flavanonol antioxidant is dihydroquercetin (DHQ).

5. The formulation of any one of claims 1-4, wherein said source of magnesium comprises magnesium lactate or magnesium citrate.

6. The formulation of any one of claims 1-5, wherein said source of L-carnitine comprises L-carnitine tartrate.

7. A formulation comprising magnesium lactate, L-carnitine-L-tartrate and a physiological acceptable carrier for use in treating a liver condition in combination with exercise.

8. The formulation of claim 7, further comprising an antioxidant which is dihydroquercetin (DHQ).

9. The formulation of claim 8, wherein the physiologically acceptable carrier is an aqueous solution.

10. The formulation of any one of claims 1-9, being formulated for oral administration.

11. The formulation according to any one of claims 1-10, wherein the liver condition is non-alcoholic fatty liver disease (NAFLD).

12. The formulation of any one of claims 1-10 for use in treating or reducing the severity of a liver condition in a subject upon exercising.

13. The formulation according to claim 12, wherein the composition is administered to the subject prior to exercising.

14. The formulation according to claim 12, wherein the liver condition is fatty liver disease, hepatitis, fibrosis or cirrhosis.

15. The formulation according to claim 14, wherein the fatty liver disease is non alcoholic fatty liver disease (NAFLD) or alcoholic liver disease (ALD).

16. The formulation according to claim 15, wherein the NAFLD is non-alcoholic steatohepatitis (NASH).

17. The formulation of claim 16, wherein the administration of the formulation is performed from 10 minutes to 3 hours prior to exercising.

18. The formulation of claim 16, wherein the administration of the formulation is performed about one hour prior to exercising.

19. The formulation of any one of claims 11-18, wherein said administration is performed once a day.

20. The formulation of any one of claims 11-19, wherein said administration is performed at a time period that ranges from 10 days to 365 days.

21. A kit comprising a packaging material and a source of magnesium, a source of L-carnitine and a physiologically acceptable carrier packaged within said packaging material, wherein the kit is for use in treating a liver condition upon exercising.

22. The kit of claim 21, wherein said source of magnesium and said source of L-carnitine are packaged individually within said packaging material.

23. The kit of claim 21, wherein each of said source of magnesium, said source of L-carnitine and said carrier are packaged individually within said packaging material.

24. The kit of claim 22, wherein said source of magnesium and said carrier are packaged together within said packaging material, the kit further comprising instruction to mix said source of L-carnitine with said source of magnesium and said carrier.

25. The kit of any one of claims 21-24, further comprising an antioxidant packaged within said packaging material.

26. The kit of claim 25, wherein said antioxidant is dihydroquercetin (DHQ).

27. A kit comprising a packaging material and the formulation of any one of claims 1-10 packaged within said packaging material, the kit for use in treating a liver condition upon exercising.

28. A nutrition supplement comprising the formulation of any one of claims 1-10.

29. The nutrition supplement according to claim 28 for use as a medicament in combination with exercise.

30. The nutrition supplement of any one of claims 28-29 for use in the treatment of a liver condition.

31. The formulation according to any one of claims 1 to 10, further comprising caffeine, a vitamin, a mineral or any combination thereof.

32. The nutrition supplement according to claim 28, further comprising caffeine, a vitamin, a mineral or any combination thereof.

33. The formulation according to claim 10, wherein said formulation is capsulated.

34. The formulation according to claim 33, wherein said formulation is administered by providing a subject with two capsules.

35. The formulation according to claim 1, wherein said formulation further comprising palatinose, citric acid, carnitine, magnesium citrate, wild berry flavoring, cranberry flavoring, ascorbic acid, sucralose, potassium sorbate and water.

36. The formulation according to claim 35, said formulation comprising 1 gr/per dose palatinose, 0.2 gr/per dose citric acid, 1.5 gr/per dose carnitine L, 1.5 gr/per dose magnesium citrate, 0.1 gr/per dose wild berry flavoring, 0.05 gr/per dose cranberry flavoring, 0.01 gr/per dose ascorbic acid, 0.012 gr/per dose sucralose, 0.0225 gr/per dose potassium sorbate and 10.6055 gr/per dose water, wherein the dose is of 15 ml.
